(19) United States
(12) Patent Application Publication
(10) Pub. No.: US 2010/0100008 A1
(43) Pub. Date: Apr. 22, 2010

(54) Substance Detecting Diaper

(76) Inventors: Zofia Chciuk, Pacific Grove, CA (US); Sally Schmidt, Salinas, CA (US); Kristine Yochim, Pacific Grove, CA (US)

Correspondence Address: LARIVIERE, GRUBMAN & PAYNE, LLP, 19 UPPER RAGSDALE DRIVE, SUITE 200, MONTEREY, CA, 93940, US

(21) Appl. No.: 12/253,109

(22) Filed: Oct. 16, 2008

(51) Int. Cl. A61B 5/00; A61F 13/15

(52) U.S. Cl. 600/584; 604/358

(57) ABSTRACT

An apparatus and method for the detection of substances in an excretion. A diaper is generally utilized and a pad containing one or more reagent strips is placed within a sleeve in the crotch of the diaper. An excretion within the diaper, such as urine, occurs and the reagent strips are activated and test for the presence of certain substances. The substances tested for are generally ones in which mothers may abuse and are harmful to and transferred to the infant, but may be any substance the presence of such is helpful to know.

### TECHNICAL FIELD

The present invention relates to the field of drug testing. More particularly, the present invention relates to an apparatus and method for testing excretions for drugs passed to infants by their drug dependent mothers.

### BACKGROUND ART

Substance abuse around the world is widespread. A significant number of substance abusers are pregnant women. The problem is so widespread that these infants born of substances abusers have obtained common names, for example, the infants of crack cocaine users have acquired the name “crack babies,” a name that has been added to the dictionary.

Infants of drug dependent women tend to have significantly more problems than infants born of women who are not so dependent. In the neonatal period, low birth weight, prematurity, infections, aspiration pneumonia, congenital malformations, hyaline membrane disease, abnormal heart rate, substance withdrawal, abnormal breathing patterns, and even mortality are increased and common among such infants.

These significant problems require that these infants receive immediate care and attention. However, before any treatment may be given, the substances must be detected, i.e., the infant must test positive for any substances passed to them by their mother.

Unfortunately, this detection is not easy. A substance abusing mother may have taken a significant amount of substances; yet, the infant may not immediately show signs of such abuse, therefore, not arousing suspicion. Further, while some mothers may admit to such substance abuse, maternal admissions, if any, of drugs used may not be accurate or trustworthy because of the legal and moral consequences of such abuse.

There have been other efforts to detect such substances of infants including assaying meconium to detect substances. However, this and other approaches may be impractical and cost prohibitive. With the growing burden of health care costs, minimizing tests and expenditures has been the normal course of action to reduce burdens to the health care system.

Therefore, what is needed is an efficient apparatus and method for detecting infants with substances to provide them the care and need necessary as early as possible, while minimizing costs.

### SUMMARY OF THE INVENTION

Accordingly, the present invention is a substance detecting apparatus and method comprising a diaper and one or more reagent strips. The reagent strips may be affixed to a pad. The diaper generally includes a sleeve in which the reagent strips may be inserted, or any other means for affixing the reagent strips in place, such as a hook and loop system, or other detachably attachment system.

The diaper is a standard diaper with, generally, a sleeve to retain the reagent strips. Like the standard diaper, the diaper used employs a crotch to collect excretions, such as fecal matter and urine, while the human is wearing the diaper. The sleeve contains one or more passages and is positioned in the crotch of the diaper. The reagent strips are placed in the crotch where they come into contact with urine, activating them.

Each reagent strip may test for substances known to be abused by women who may become pregnant. These substances may be marijuana, methamphetamine, cocaine, opiates, phenethlamines, phencyclidine, and amphetamine, among others.

When a reagent strip is activated, it will indicate the positive or negative presence of one or more substances. The care facility may employ confirmatory testing should an infant test positive for a substance, as the reagent strips may show a false positive.

The cotton pad the reagent strips are coupled to may be used for confirmatory testing by submitting the cotton pad to a laboratory. This is beneficial because obtaining the necessary urine for confirmatory testing may be difficult, e.g., because of fecal contamination. Further, because delays in confirmatory testing may arise or the need for such confirmatory testing may not be immediately known, it is beneficial to have an already obtained sample on hand.

In another embodiment the present invention may be useful detecting other substances, such as beneficial substances, or other substances not necessarily arising from the mother's use of a substance.

Finally, in adults suffering from incontinence who also wear diapers like undergarments, the present invention may be useful for monitoring assimilation of medicines administered by caregivers, especially where the patient is unable or unwilling to accurately report their medicine intake.

### DESCRIPTION OF THE DRAWINGS

FIG. 1 is a prospective illustration of an embodiment of the current invention.

FIG. 2 is a prospective illustration of an embodiment of the current invention.

FIG. 3 is a drawing of the reagent strips connected on to a pad.

### DETAILED DESCRIPTION OF SELECTED EMBODIMENTS

The present invention is drawn to an apparatus and method for testing excretions for substances. This generally involves testing the urine of humans for the substances. This present invention provides an early indication of substance presence to allow an early, appropriate response.

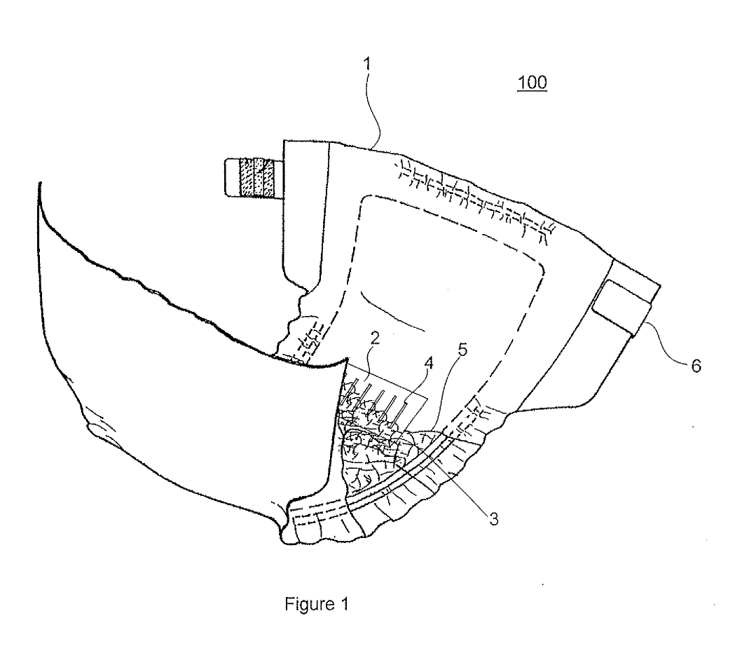

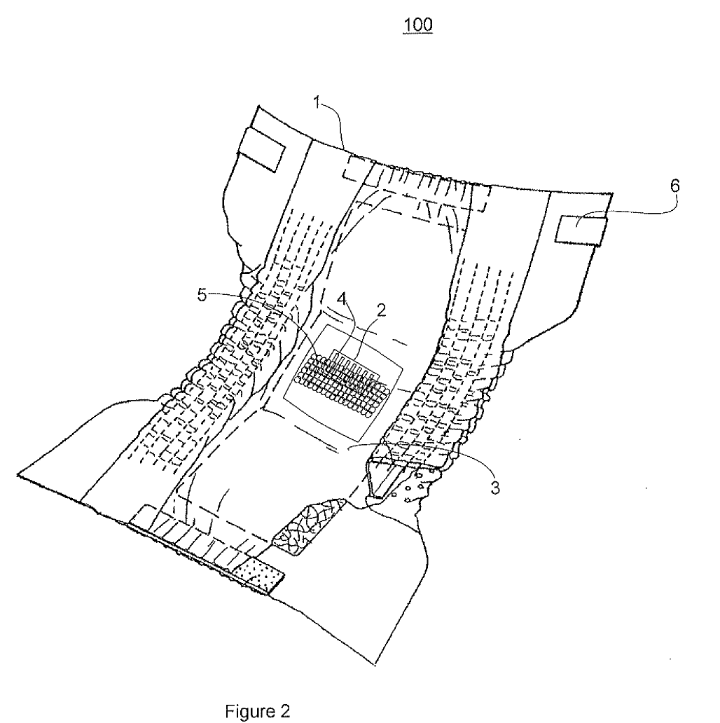

As shown in FIG. 1-2, the apparatus 100 comprises diaper 1. Diaper 1 has crotch 3 and fasteners 6. Diaper 1 encircles the human (not shown). Diaper 1 has crotch 3. Crotch 3 has sleeve 5.

Pad 2, shown in FIG. 1-3, contains one or more reagent strips 4. Pad 2 may be made of cotton or a similar material that absorbs liquids. Reagent strips 4 contain reagent strip labels 7. Pad 2 may be inserted into sleeve 5 in crotch 3 of diaper 1. While inserted into sleeve 5, pad 2 and, therefore, correspondingly reagent strips 4, may come into contact with urine (not shown) of the human. This urine then activates the reagent strips to test for the presence of a specified substance.

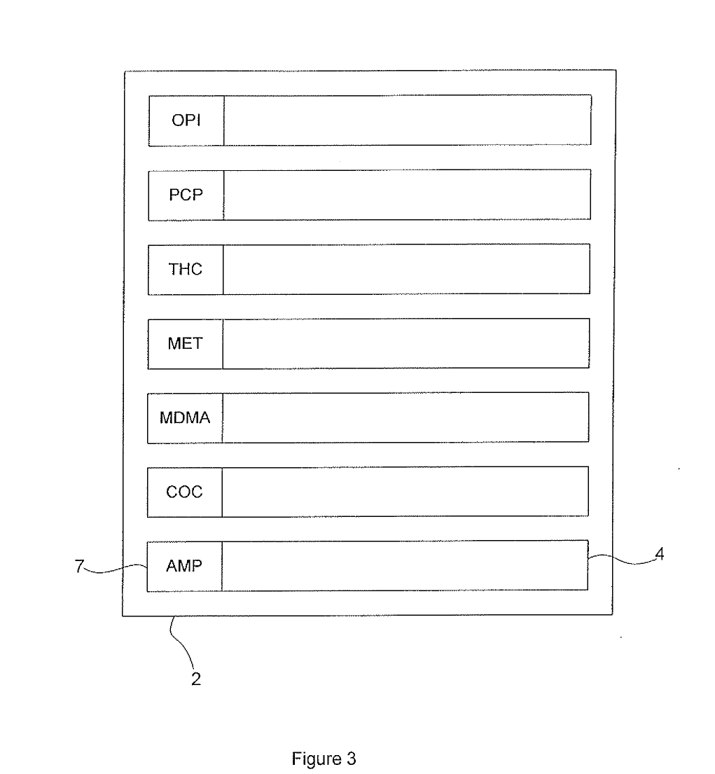

The reagent strip 4 may detect marijuana, methamphetamine, cocaine, opiates, phenethlamines, phencyclidine, amphetamine, or any other substances, such as beneficial ones. The substance the reagent strip 4 detects for may be indicated on the reagent strip label 7, as shown in FIG. 3. If a reagent strip 4 indicates a positive for the presence of a substance, a confirmatory test may be partaken in.

Pad 2 may be detachably attached to reagent strips 4 and utilized for primary, confirmatory or secondary testing. For example, pad 2 may be placed in a vile and stored for future use if needed or submitted to a laboratory wherein said laboratory tests urine obtained from pad 2 for the presence of a specified substance.

Diaper 1 may be disposable or non-disposable. Further, diaper 1 may by cotton. Fasteners 6 may be an adhesive or a hook and loop cooperating system, such as Velcro.

Another embodiment may be directed at detecting other substances using reagent strips 4, such as beneficial substances or other substances not necessarily arising from the mother's (not shown) use of a substance.

In another embodiment, reagent strips 4 disposed in the crotch 3 of diaper 1 may detect medicines administered by a caregiver (not shown) to an adult (not shown) wearing apparatus 100 to determine the assimilation of administered medicines.

Diaper, as contained herein and as used in the claims, is to mean any absorptive material, generally used as, but need not be used as, an undergarment placed about the genitals. This may be an infant's diaper, an adult diaper, an incontinence pad, or any other device, system or apparatus of similar nature.

Information as herein shown and described in detail is fully capable of attaining the above-described object of the invention, and is, thus, representative of the subject matter which is broadly contemplated by the present invention. The scope of the present invention fully encompasses other embodiments which may become obvious to those skilled in the art, and is to be limited, accordingly, by nothing other than the appended claims, in which reference to an element in the singular is not intended to mean “one and only one” unless explicitly so stated, but rather “one or more.”

All structural and functional equivalents to and combinations of the elements of the above-described preferred embodiment and additional embodiments that are known to those of ordinary skill in the art are hereby expressly incorporated by reference and are intended to be encompassed by the present claims. However, it should be readily apparent to those of ordinary skill in the art that various changes and modifications in form, apparatus material, and fabrication material detail may be made without departing from the spirit and scope of the invention as set forth in the appended claims.

Moreover, no requirement exists for a device or method to address each and every problem sought to be resolved by the present invention, for such to be encompassed by the present claims. Further, no element, component, or method step in the present disclosure is intended to be dedicated to the public regardless of whether the element, component, or method step is explicitly recited in the claims. No claim herein is to be construed under the provisions of 35 U.S.C. §12, sixth paragraph, unless the element is expressly recited using the phrase “means for.”

### INDUSTRIAL APPLICABILITY

The current invention is applicable to drug testing of humans.

We hereby claim:

1. An apparatus for detecting one or more substances present in an excretion comprising:
a diaper having a crotch;
one or more reagent strips, each of said reagent strips for detecting a substance;
said reagent strips disposed in said diaper's crotch.

2. The apparatus of claim 1 further including:
a sleeve having a one or more passages for housing said reagent strips.

3. The apparatus of claim 1 wherein said excretion is urine.

4. The apparatus of claim 1 further comprising:
a pad;
said reagent strips detachably attached to said pad.

5. The apparatus of claim 4 wherein said pad is composed of cotton.

6. The apparatus of claim 1 wherein said reagent strips detect the presence of one or more detrimental substances.

7. The apparatus of claim 1 wherein said reagent strips detect the presence of one or more of the substances: marijuana, methamphetamine, cocaine, opiates, phenethlamines, phencyclidine, and amphetamine.

8. The apparatus of claim 1 wherein said reagent strips detect the presence of one or more beneficial substances.

9. A method for detecting one or more substances present in an excretion comprising the step:
installing one or more reagent strips in the crotch of a diaper.

10. The method of claim 9 wherein said installing is by disposing said reagent strips in a sleeve wherein said sleeve contains one or more passages.

11. The method of claim 9 wherein said excretion is urine.

12. The method of claim 9 further comprising:
coupling said reagent strips to a detachably removable pad.

13. The method of claim 12 further comprising:
testing said pad for the presence of a specified substance.

14. The method of claim 9 wherein said reagent strips detect the presence of a detrimental substance.

15. The method of claim 9 wherein said reagent strips detect the presence of one or more of the substances: marijuana, methamphetamine, cocaine, opiates, phenethlamines, phencyclidine, and amphetamine.

16. The method of claim 15 further comprising the step of:
analyzing results of said detecting.

17. The method of claim 9 wherein said reagent strips detect the presence of a beneficial substance.

18. The method of claim 9 further comprising the step of:
coupling said reagent strips to a pad;
activating said reagent strips with said excretion;
absorbing said excretion by said pad;
testing said reagent strips for the presence of one or more of the substances: marijuana, methamphetamine, cocaine, opiates, phenethlamines, phencyclidine, and amphetamine.

19. The method of claim 18 further comprising:
performing a confirmatory test of said testing by testing said pad for the presence of the specified substance.

20. An apparatus for detecting one or more substances present in an excretion comprising:
a diaper with a crotch;
a sleeve containing one or more passages wherein said sleeve is disposed in diaper's crotch;
one or more reagent strips detachably attached to a pad;
said reagent strips disposed in said sleeve.
